(19) United States
(12) Patent Application Publication
(10) Pub. No.: US 2002/0086010 A1
(43) Pub. Date: Jul. 4, 2002

(54) Adding heat-sensitive biologically active material to food or cosmetic compositions

(76) Inventor: Woodrow C. Monte, Tempe, AZ (US)

Correspondence Address: Squire, Sanders & Dempsey L.L.P., Two Renaissance Square, 40 North Centeral Avenue, Suite 2700, Phoenix, AZ, 85004-4498, US

(21) Appl. No.: 09/747,078

(22) Filed: Dec. 21, 2000

(51) Int. Cl. A61K 39/395; A61K 38/43

(52) U.S. Cl. 424/130.1; 424/94.1

(57) ABSTRACT

Disclosed herein is a method for making a composition, such as a food or cosmetic composition, including a biologically active material, such as an enzyme or antimicrobial. The method includes the steps of insulating the active, heating the composition to a temperature that would normally denature or destroy the active, and adding the active to the composition at the denaturing temperature. The active is in a form that enables a beneficial amount to survive and remain active in the composition.

### FIELD OF THE INVENTION

This invention relates to a method for adding one or more heat sensitive, biologically active materials (such as lactase enzyme) to a composition, such as a food or cosmetic composition, at an elevated temperature that would normally destroy or denature the active material(s).

### BACKGROUND OF THE INVENTION

As used herein, the term “active” or “actives” refers to one or more biologically active substance(s), material(s) or constituent(s) and includes enzymes, antibodies, vitamins or a combination thereof. As used herein, the term “composition” refers to any substance, intermediate, constituent or formula in which an active may be included to impart a beneficial effect, and includes foods, cosmetics and skin lotions. As used herein, the term “food” or “foods” means any foodstuff suitable for human or animal consumption, or intermediate composition or ingredient used to make a foodstuff, and includes liquids. As used herein, the term “denature” or “destroy,” when used in relation to an active, means that the active has lost its beneficial biologically active properties. As used herein, the term “denaturing temperature” refers to a temperature capable of destroying or denaturing the relevant active. As used herein, the term “beneficial effect” means a measurable desired change to a composition due to the presence of an active added to the composition. As used herein, the term “container” refers to any vessel or structure used to package a composition.

Many compositions according to the invention are heated during processing to or above the denaturing temperature of an active that may be added to the composition. Presently any such active must be added to the composition while the composition temperature is below the denaturing temperature (either prior to or after the composition has been heated to the denaturing temperature) to achieve a beneficial effect. If added beforehand, the active is denatured when the composition containing the active is heated to or above the denaturing temperature.

For example, some compositions must be heated to a temperature of 180° F. or higher to destroy potentially harmful microorganisms. Some actives are denatured at such temperatures and must be added at a temperature below the denaturing temperature and allowed time to work at that lower temperature in order to impart a beneficial effect. Because the efficacy of an active in a composition is based, at least in part, upon (1) the amount of the active added to the composition, and (2) the time the active remains in the composition without being denatured, either a relatively large amount of active must be added to a composition (especially when the active is added prior to the composition being heated to or above the denaturing temperature), or the time the active remains in the composition must be relatively long, or both. Processing may therefore be expensive because the active is often costly and/or the extra manufacturing and handling time and procedures required to allow the active time to work are costly.

In one specific example, a lactose-converting enzyme (called lactase enzyme) may be added to a composition to convert lactose to another substance, such as a monosaccaride. A small amount of enzyme converts only a small amount of lactose in a given time period, but if allowed enough time, will convert nearly all of the lactose in the composition. Therefore, the greater the amount of enzyme added to the composition, the faster the lactose is converted, and the longer the time the enzyme remains in the composition the greater the amount of lactose converted. The problem is that lactase enzyme is expensive, and manufacturing time is expensive. It would therefore be desirable to add only a small amount of lactase enzyme, to keep manufacturing times short, but to still obtain a significant beneficial effect. This could be accomplished by adding a small amount of enzyme to the composition immediately prior to a composition being sealed in a container so the enzyme could work in the container during storage and shipping. Until now this was not a viable option for many compositions because, in order to kill microbes, the compositions were heated to and packaged at a temperature that denatured the active and/or heated to the denaturing temperature while sealed in the container. This process is sometimes called hot-pack processing. The composition is processed hot; put in the container hot; and sealed hot.

In a specific example in which lactose is converted to another substance, lactase enzyme is added to milk prior to the milk being pasteurized and reaching the denaturing temperature in order to reduce the lactose content of the milk. The lactose-converting enzyme must thus be added prior to the composition reaching the denaturing temperature and is normally added to the milk before processing begins. It generally takes between 6 and 24 hours for the enzyme to convert between 70% and 100% of the lactose and the milk composition must be cooled during this time. Therefore, the process is relatively expensive because a relatively large amount of enzyme (which costs approximately $200/lb) is used, additional manufacturing time is required to allow the enzyme to work, and the refrigeration process requires energy, which is costly.

Another method, called membrane filtration, has been used to separate lactose from protein in whey. The process is relatively expensive and involves semi-permeable membranes to filter the protein molecule (which is a large molecule) from lactose (which is a small molecule). The protein is dried or refrigerated afterwards and this low-lactose whey protein is used to manufacture medical food, thus lowering the amount of lactose in the medical food. Generally, the whey is passed through a membrane one or more times until a desired level of lactose reduction is attained.

Currently, one option for companies desiring to offer low-lactose medical food is to purchase whey protein that already had much of the lactose removed (usually about 70% or more) by the membrane filtration method. However, such protein costs between approximately $1.80 and $4.00 per pound. In contrast, significant savings could be achieved using liquid whey protein concentrate having approximately 20% protein content by weight and 50-75% lactose content by weight, which costs as little as approximately $0.20 per pound. In one example, approximately 10% of the weight of an enteral medical food composition is protein, and a typical serving of such a medical food is 8 ounces. So, assuming the protein used in such a medical food currently costs $3.00 per pound, the protein in an 8-ounce serving costs about $0.15. If protein costing $0.20 per pound could be used instead, the cost of protein per serving would be approximately $0.01, yielding a savings of roughly $0.14 per serving.

Another method for utilizing an active in a composition involves reducing the lactose content of a dairy product, such as ice cream. The method generally comprises adding lactase enzyme (which converts lactose to sugar) to the dairy product and then heating the dairy product to a temperature in excess of about 140° F. (which is below the denaturing temperature) for about three hours. This allows the enzyme to hydrolyze the lactose in the dairy product. There are disadvantages associated with this method. First, relatively large amounts (about 200 to 1000 parts per million) of the lactase enzyme are again required because the conversion of the lactose enzyme must occur prior to the pasteurization of the dairy product (which heats the product to an elevated temperature that denatures the enzyme). Second, the step of heating the composition to 140° F. for about three hours is expensive.

In another method, lactase enzyme is added to a dairy composition at a low temperature in order to prevent the growth of microorganisms. Maintaining dairy products at refrigerated temperatures, however, is an added cost to any dairy operation. Further, often only a 70% enzymatic reduction of the lactose is achieved utilizing this method. Additionally, adding the enzyme at a relatively low temperature after the pasteurization process may introduce unwanted microbes into the composition.

It is also known for people to ingest active enzymes, although the enzymes are not included as part of a composition. For example, Bean-O is an enzyme people take to reduce or eliminate flatulence.

Accordingly, it would be desirable to provide a method that reduces the amount of active, such as lactase enzyme, required to achieve a beneficial effect in a composition and/or that produces a composition containing one or more actives that are ingested thus giving the active the opportunity to work in an individual's digestive tract. As added benefits, it would be desirable if the beneficial effect on the composition were greater than the effect using known methods and if overall manufacturing costs were reduced.

### SUMMARY OF THE INVENTION

One aspect of the invention is a method for adding a structure or device including an active to a composition wherein the structure or device temporarily prevents at least some of the active from coming into direct contact with the composition, thus enabling the active to provide a beneficial effect. The method may be utilized with any type of composition, such as a food or cosmetic composition that (1) is heated during processing to or above the denaturing temperature of an active, and (2) could utilize the active to provide a beneficial effect. The structure or device is added to the composition (a) when the composition is at or above the denaturing temperature of the active, or (b) when the composition is below the denaturing temperature of the active, and is afterwards heated to or above the denaturing temperature. Another aspect of the invention is a structure or device, such as a tablet, capsule or overlay including an active. The device may be utilized in any composition in which the active could be used to provide a beneficial effect, regardless of when it is added. Among the beneficial effects that may be provided are lactose conversion or the addition of any active for ingestion whereby it provides a benefit by being in the digestive system.

Preferably, the active is included as part of a structure or device, such as a tablet, capsule or overlay wherein at least enough of the active is protected, either by being coated, covered or otherwise protected, to prevent the active from coming into direct contact with the composition until the structure or device at least partially dissolves. In the embodiment most preferred, the device is produced by pressing powdered active together with one or more other powdered materials to create a tablet, and the tablet is then preferably coated with a sugar coating to form a coated tablet. The coating keeps the coated tablet from dissolving too quickly in the composition and thus helps to keep at least some of the active from coming directly into contact with the composition until (it is believed) the composition temperature falls below the denaturing temperature. While the invention is not limited to the theory upon which it may work, it is believed that the active in the tablet may (and probably does) reach the elevated temperature, but by remaining dry, it is not denatured. This is thought to occur because certain actives, such as enzymes, are more active when expressed in a liquid and are more prone to being denatured while in that state.

The structure or device may be used in the method of the invention, or used in other methods whereby the composition is not heated to or above the denaturing temperature of the active added to the device.

Therefore, the function of the method of the invention is to either (a) add an active to a composition at or above a temperature that would normally denature the active, wherein some or all of the active is not denatured, or (b) add an active to a composition and then heat the composition to or above a temperature that would denature the active, wherein some or all of the active is not denatured. The way this function is achieved is by including the active as part of a structure or device wherein at least some of the active is covered, coated or otherwise protected so that it does not directly contact the composition until the structure or device at least partially dissolves. The result is that the active imparts a beneficial effect to the composition. Each of the terms “structure” and “device” are defined herein to include any structures or devices that can be used to practice the invention and hereinafter shall be referred to collectively as “device.”

One benefit of the method of the invention is that a much smaller amount of active, such as an enzyme, may be utilized. For example, prior art lactose-removal processes typically require from 200 to 1000 parts per million of enzyme for dairy products like whole or skim milk, each of which contains about 4.5% by weight of lactose (human milk contains about 10% lactose by weight). While the process of the invention can, if desired, utilize such high concentrations of enzyme, ordinarily a much smaller amount, such as 1 to 150 parts per million, and preferably 5 to 75 parts per million, of enzyme is employed.

As an additional benefit, high lactose protein (such as the previously-described whey protein concentrate, in either liquid or dried form) could (1) be used in place of expensive low-lactose protein and/or (2) theoretically be used to replace some or all of the sugar in a composition (assuming that sugar were to be added to the composition) if the present invention were used because some lactose-converting enzymes convert lactose to a monosaccaride such as glucose.

Another benefit of the method of the invention is that the active has a longer time to function while in the composition and a greater beneficial effect may be achieved. For example, some prior art lactose reduction procedures achieve only a seventy percent reduction of the lactose in a composition, whereas the process of the invention may eliminate all or nearly all of the lactose in a composition at a relatively low cost. Utilizing the method of the invention, the lactose hydrolyzing enzyme continues to function after the composition is packaged, permitting the enzyme to continue gradually to hydrolyze lactose over a relatively long time period. Therefore, if an active is lactase enzyme and the composition is a medical food, inexpensive whey protein concentrate can be used because the enzyme will have time to convert the lactose utilizing the process of the invention. Generally, whey protein concentrate or whey itself (which has about 85% lactose solids and about 15% mixture of minerals and protein solids) could be used.

Another benefit of the invention is that the active is not denatured and may be ingested in its chemically active form. It may be beneficial to ingest a chemically active substance because the active could function to destroy, for example, bacteria, lactose or other substances already in the in the stomach and gastro-intestinal tract and/or substances ingested afterwards.

Another benefit of the invention is that a composition need not be heated or cooled for extended times after the active is added to enable the active to function as desired. Instead, normal processing procedures for the composition can be used.

### BRIEF DESCRIPTION OF THE DRAWING

FIG. 1 is a cross-sectional view of a preferred embodiment of a tablet according to the invention, wherein the tablet includes a heat-sensitive, biologically active material.

FIG. 1B is a cross-sectional view of a preferred embodiment of a tablet according to the invention that does not include an exterior coating.

FIG. 2 is a cross-sectional view of a capsule according to the invention.

FIG. 3 is a partial cross-sectional view of a container including an overlay.

FIG. 4 is a block diagram generally depicting a method of manufacturing a composition that shows one possible way in which a heat-sensitive, biologically active material may be added.

### DETAILED DESCRIPTION OF A PREFERRED EMBODIMENT

The invention generally includes (1) a device including an active, and (2) a method of adding an active to a composition without denaturing at least some of the active. The method includes the step of adding a device including an active to a composition that reaches a temperature that would normally denature the active. The composition may be at the denaturing temperature (or higher) when the device including active is added and/or at any time after a device including active is added. The device may be added (a) while the composition is at or above a temperature that would denature the active, or (b) when the composition is below the denaturing temperature of the active, and is afterwards heated to or above the denaturing temperature.

The active is preferably contained within, or is otherwise part of, a device such as a tablet, capsule or overlay. A device according to the invention preferably includes (1) one or more actives, and optionally one or more of the following (2) one or more antimicrobial agents, (3) one or more fillers, (4) one or more edible acids, (5) one or more buffers, and (6) an exterior coating, such as a sugar coating.

As previously stated, a composition according to the invention includes, but is not limited to, foods, cosmetics and skin lotions. Some compositions that may be used to practice the invention are disclosed in U.S. Pat. Nos. 4,931,300, 4,112,123, 5,156,873, 5,389,391 and 5,614,241, the respective disclosures of which that are not inconsistent with the disclosure in this application are incorporated herein by reference. Among the foods in which the invention may be used are (1) medical foods, such as enteral foods for hospital patients, (2) nutritional drinks, such as those used in nursing homes and hospitals, (3) shake drinks, especially those fortified with vitamins, minerals and/or carbohydrates and sold as sports drinks, or (4) other fortified sports drinks (such as GATORADE-type drinks) or juices. A food may also be an animal food, or an intermediate, such as a protein-containing intermediate, used in the manufacture of any food. Many of these compositions are pasteurized or otherwise heated to kill microbes and/or hot packed to prevent microbes from contaminating or recontaminating them. As used herein, the term “microbes” includes bacteria.

The process of the invention is preferably used in connection with acidic (low pH) compositions because a different kind of bacteria, which requires higher temperatures over a longer period to kill, can grow in more neutral pH compositions. Normally, a low pH (preferably between 4.5 and 6.0) alone inhibits the growth of many bacteria, and the ones that will grow in a low pH medium are destroyed with hot packing. But the heat of hot packing, which generally involves heating the composition to about 180°-190° F. for a relatively short period of time, would destroy many beneficial actives, such as many antibodies and enzymes, and would either destroy or reduce the efficacy of other actives, such as certain vitamins and antioxidants.

The active may be one or more enzymes, antibodies, vitamins, minerals, carbohydrates, medicines, or a combination of one or more of the above. By way of example, and not limitation, among such enzymes is powdered lactase (fungal lactase enzyme, for example). If the composition is one to which a lactase enzyme is added, the invention preferably removes preferably between 70 and 95% of the lactose from the composition. Other enzymes that may be utilized in the practice of the invention, include but are not limited to, those that (1) facilitate digestion and use by the human body of foods and/or food components such as vitamins, minerals, or (2) promote the cleansing of teeth and the oral cavity. For example, but not by way of limitation, such enzymes can include alpha amylase, protease, beta glucanase, glucoamylase, glucose oxidase, pectinase, xylanase, or other protein hydrolyzing enzymes, starch dextrinizing enzymes, starch saccharifying enzymes, and/or cellulose hydrolyzing enzymes. There is also a possibility that diabetes may be induced by bovine serum albumin present in some infant formulas. Using the invention, an enzyme may be added to the infant formula that would destroy bovine serum albumin, or greatly reduce its ability to cause problems by the time the formula was consumed.

The active may also be one or more antibodies, either alone or in combination with other actives. For example, antibodies used to fight colitis or other afflictions caused by a bacterium causing problems in the digestive system, such as inflammation of the colon, may be used as actives or be among the actives. Antibodies that fight the types of bacteria found in the gastro-intestinal tract, such as staph bacteria, may also be used, as could an antibody that fights a bacterium that causes ulcers.

Colostrum could also be an active utilized in the invention. Colostrum is a substance contained in milk that contains antibodies to fight harmful bacteria and colostrum can be created to fight specific bacteria. For example, cows can be infected with certain bacteria while they are pregnant, such as harmful bacteria that may be present in the gastrointestinal tract of humans. When the calf is born it would be fed artificial milk replacer and the cow's milk, with the colostrum containing the antibodies beneficial to humans, is removed from the cow. The weight of active antibody within colostrum can be up to 10% by weight of the dry weight of the colostrum. Such colostrum, along with the valuable antibodies, could be a food supplement but the antibodies are very heat sensitive. The process of the invention allows colostrum to be introduced to a composition while the composition is still at or above the denaturing temperature of the antibodies.

One or more vitamins may also be used as actives, with or without other actives. Such vitamins include: (1) vitamin A, a fat-soluble aliphatic alcohol, C₂OH₂₉OH, found in substances such as fish-liver oil, egg yolk, and butter; this vitamin occurs in two forms, vitamin A₁ and A₂, (2) vitamin B complex), a group of unrelated water-soluble substances including: (a) B₁ (thiamine); (b) vitamin B₂ (riboflavin); (c) vitamin B₆ (pyridoxine); (d) nicotinic acid; (e) pantothenic acid; (f) biotin (also called vitamin H); (g) inositol; (h) choline; (j) folic acid; and (k) vitamin B₁₂ (cyanocobalamine), (3) vitamin C, an organic compound C₆H₃O₆, occurring in citrus fruits, tomatoes and various vegetables, (4) vitamin D, which is any of several related vitamins occurring in substances such as fish-liver oils, milk, egg yolks, and includes (a) vitamin D₁, a mixture of calciferol with another sterol prepared by the ultraviolet irradiation of ergosterol; (b) vitamin D₂ (calciferol); (c) vitamin D₃, a substance similar to vitamin D₂, found chiefly in fish liver oils, (5) vitamin E, a substance consisting of a mixture of tocopherols, (6) vitamin K, a vitamin occurring in certain green vegetables, fish meal, hempseed and other substances, including (a) vitamin K1, found chiefly in alfalfa leaves, and vitamin K2, found chiefly in fish meal, and (7) vitamin P, a mixture of the flavones occurring especially in citrus juice and paprika.

If one of the actives to be added was a vitamin and/or antioxidant a potential added benefit to the composition would be that a relatively small amount would be added to the composition since the heat destruction that normally occurs during processing would be diminished or eliminated. This relatively small amount may not cause flavor degradation of the composition, which can occur if a relatively large amount of vitamins and/or antioxidants were added.

One or more phytochemicals can also be used as actives. Phytochemicals include sulphoraphane, PEITC (phenethylisothiocyanate), indole-3-carbinol, aurones, chalcones, anthocyanidins, flavanones, anthocyanidins, flavones, flavonols, flavan 3-ols, oligomeric flavonoids, biflavonoids, isoflavonoids and other compounds which are, prior to a plant being harvested, stored in the epidermal cells of the plant and which typically absorb light having wavelengths in the range 10 to 800 nm.

One or more alimentary vegetable compositions can also be used as actives. As used herein, the term alimentary vegetable composition means a vegetable or a part of a vegetable that alleviates, prevents, or remedies an impairment of the normal state of a human being. The impairment interrupts or modifies the performance of the vital functions of the human being, such impairment being a response to a specific infective agent (e., worms, bacteria, or viruses) or a combination of such factors. A vegetable is an organism typically characterized by lack of locomotive movement (absence of locomotion and of special organs of sensation and digestion) or rapid motor response. Vegetable parts can be produced by any desired means including without limitation, (1) mechanical means, such as, for example, by grinding or cutting leaves or seeds or stems to produce a vegetable part comprised of such cut or comminuted leaves or seeds, (2) chemical means, such as by extraction by boiling vegetable part(s), such as seeds, stems or leaves in water to extract a particular component from the plant part(s), or (3) by contacting a vegetable part such as, for example, a leaf, with a liquid, gaseous, or dry composition which interacts with the part to extract and/or separate a particular component or components. Alimentary vegetable compositions are medicaments and do not generally include vegetables or vegetables parts that function only as food. Examples of alimentary vegetable compositions include (1) cayenne powder made from drying and grinding the whole seeds of the vegetable, (2) Capsicum frutescens longum; a sedative powder made from drying and grinding the root of the vegetable, (3) Valeriana officinalis; a powder made the flow heads of vegetables of the genus, (4) Anthemis, which is flower heads containing a diaphoretic and antispasmodic composition (5) an antiseptic oil containing thymol and carvacrol that is produced from vegetables of the genus Thymus, (6) an aqueous solution produced by boiling the vegetable Nepeta Cataria, (7) a member of the family Labiatae in water, and, (8) an oil obtained by the stream distillation of a vegetable of the genus Allium.

If more than one active is to be used in a composition, depending upon their respective compositions, the actives may be first mixed in dry or wet form, admixed, dissolved, or dispersed in a dry carrier, or in water, ethanol, liquid, or gas carrier, and dried, in order to form an active for adding to a composition. The active may be part of the core or a coating of a device according to the invention.

If desired, a powder filler like maltodextrin, or some other carbohydrate may be included as part of a structure or device according to the invention. If used, the powder filler could comprise from 0.1% to 75% by weight of the structure or device. The purpose of the filler may be to bind the structure or device together, or to cover, coat or otherwise protect the active or to reduce the amount of active (some of which are expensive) used.

A device according to the invention may also include one or more antimicrobial agents along with the active(s). If used, the antimicrobial agent(s) could be mixed with the active(s) or other components. Some preferred antimicrobial agents include sorbic acid, benzoic acid, sodium benzoate, potassium sorbate, sodium sorbate, and potassium benzoate.

A structure or device according to the invention may also include an edible acid along with the active(s) in order to adjust the pH of the composition. If used, the edible acid(s) could be mixed with the active(s) or other components. If an edible acid were included, it would preferably comprise approximately 4% to 20% by weight of the structure or device. The edible acid could, for example, be malic acid, acetic acid, citric acid, lactic acid, sodium acetate, fumaric acid, and/or an acidic salt such as sodium acetate. The acid assists in adjusting the pH of the composition to an acidic range to help prohibit microbial activity.

One or more buffers may optionally be included in the device along with the active(s). If used, the buffer(s) could be mixed with the active(s) or other components. If added the one or more buffers are preferably added at 2% to 20% by weight of the entire device. The buffer is preferably a salt, and, if used, functions to help maintain the pH in the acidic range. Examples of buffering salts include anhydrous disodium phosphate, dihydrated disodium phosphate, dipotassium phosphate, sodium citrate, potassium salts, calcium salts, and/or sodium salts.

One or more of the optional materials, such as the antimicrobial agents, fillers, acids or buffers, may cover, coat or otherwise protect the active(s), or may assist in covering, coating or otherwise protecting the active(s).

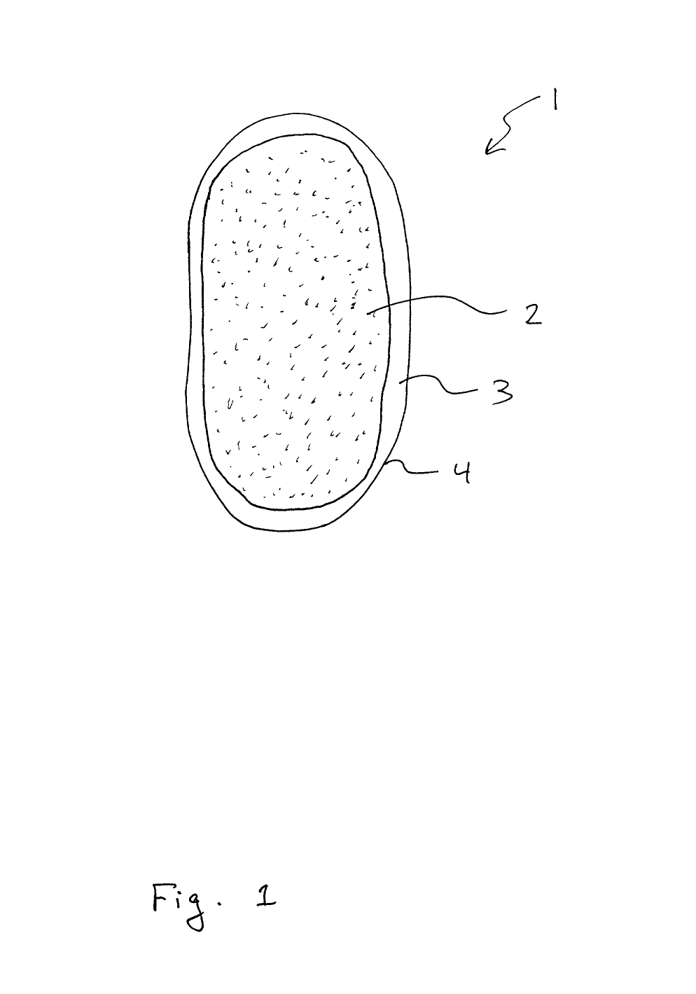

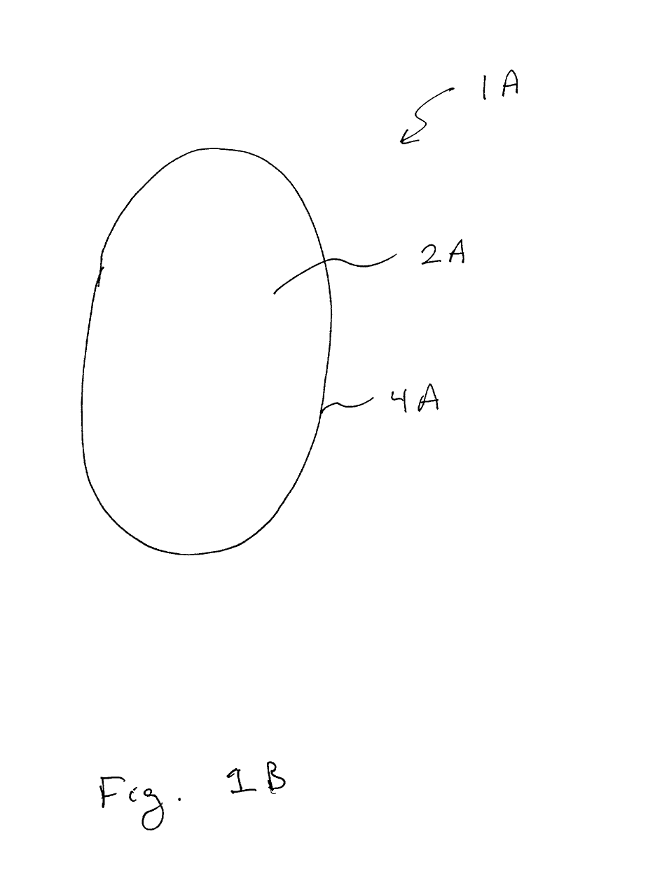

As shown in FIG. 1, a device according to the invention may be tablet 1. Tablet 1 preferably has a core 2, a coating 3 and an outer surface 4. As used herein, the term “tablet” refers to any device that comprises compressed and/or coated material, and includes pills and pellets. Tablet 1 may be of any size or shape and is preferably of a size and shape suitable to be placed inside a container in which the composition is packaged. Core 2 is the inner portion of tablet 2 and preferably includes active(s) and optionally one or more components. Preferably, the components forming core 2 are provided initially in powdered form and compressed to form core 2. Outer surface 4 is the outer most surface of tablet 1 and, if tablet 2 has an exterior coating 3, the outer surface of exterior coating 3 is the outer surface 4 of tablet 1, it being understood that tablet 1 need not have an exterior coating 3. As shown in FIG. 1B, tablet 1A has an inner core 2A and an outer surface 3A, but no exterior coating. Additionally, exterior coating 3 may be comprised of multiple layers or coatings.

The active contained in tablet 1 is at least partially coated, covered or otherwise temporarily protected from coming into direct contact with a composition in which it is placed in order to enable the active to impart a beneficial effect. Preferably, at least some of the active is in core 2 of tablet 1 and is completely covered or coated with a material such as one of the previously described fillers, buffers, edible acids, or additional active, it being understood that additional active could also sufficiently cover, coat or otherwise protect the active in tablet 1 or 1A.

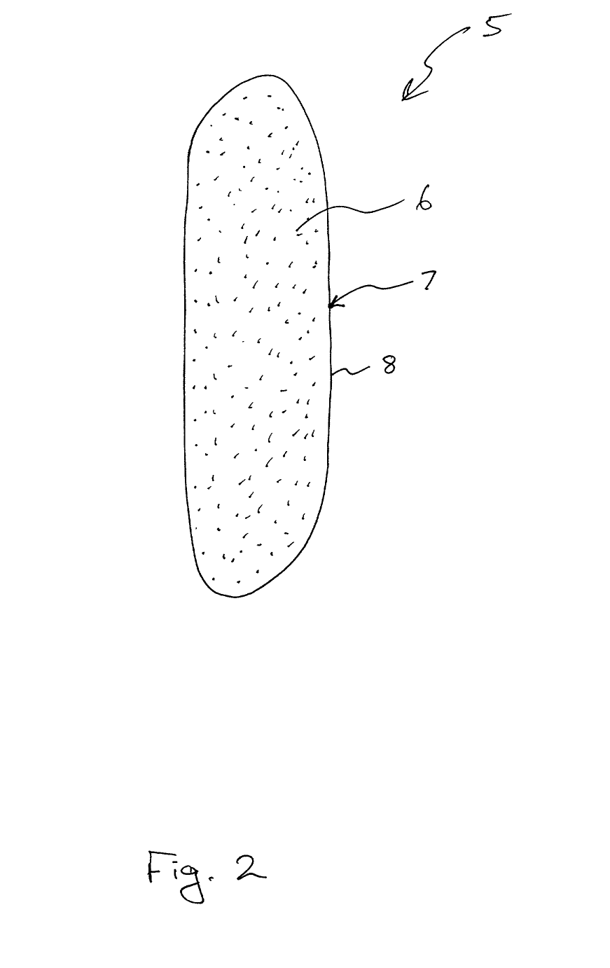

FIG. 2 shows a capsule 5 having an interior 6, a shell 7 and an outer surface 8. As used herein, the term “capsule” refers to any container or case, of any size or configuration, including an active wherein at least some of the active is covered, coated or otherwise temporarily protected from coming into direct contact with a composition into which the capsule is placed in order to enable the active to impart a beneficial effect. The exterior of a capsule is referred to herein as a “shell.” Active(s) and optionally other materials are included in interior 6. Shell 7 may be produced of any material suitable for assisting in covering, coating or otherwise protecting the active(s) in interior 6.

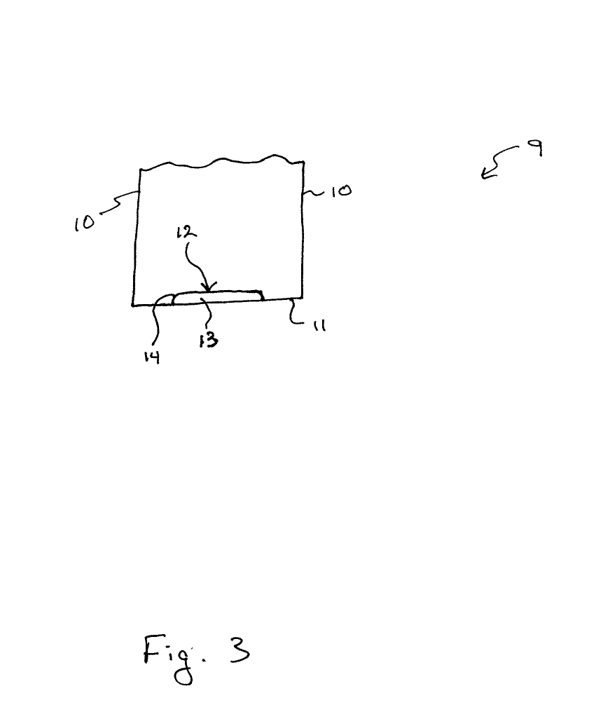

FIG. 3 shows a container 9 having a cylindrical sidewall 10 and a bottom 11. Container 9 is used for packaging a composition, and can be of any size, shape or composition suitable for that purpose. An overlay 12 is positioned on the interior surface of bottom 11. Overlay 12 has an interior 13 and an outer surface 14. As used herein, the term “overlay” means one or more layers, of any size, thickness or configuration, of material(s) placed upon a surface wherein the material(s) includes an active and at least part of the active is covered, coated or otherwise temporarily protected from coming into direct contact with a composition when the composition comes into contact with the overlay, in order to enable the active to impart a beneficial effect. As used herein, the term overlay includes films, carriers and inserts, any of which may be positioned on the inner wall of the container, or the lid of the container, in which the composition is packaged. Active(s) is contained within interior 13, which optionally includes one or more other compositions. Optionally, overlay 12 could include an exterior coating (not shown), such as a sugar coating.

If a device according to the invention does not include an exterior coating it is preferably compressed using substances that provide adequate adhesion to keep the device from dissolving too quickly when exposed to a composition at or above the denaturing temperature, in order to keep at least some of the active(s) from being denatured and enable it to provide a beneficial effect.

If the device, such as a tablet or overlay, has an exterior coating, it is preferably a sugar coating. The coating syrup may be prepared by blending water (24% by weight), starch (0.9% by weight), and fine granulated sugar (75.1% by weight) until the coating syrup has a Baume value between 33 and 37. Some coating formulations that may be used in the invention are disclosed in U.S. Pat. No. 5,578,336, the disclosure of which from col. 6, 1. 25 to col. 7,1. 19 is incorporated herein by reference. The coating can be applied in any manner, including spraying. Preferably, flavoring agents, such as those disclosed in U.S. Pat. No. 5,578,336, would not be used. Further, the active itself may be included as part of the exterior coating.

Whether or not the tablet has an exterior coating, the outer surface of the finished tablet is preferably treated with gamma rays to reduce the number of microbes present.

In lieu of a tablet, capsule or overlay, other devices for coating or covering an active may be utilized. A device (such as a tablet or capsule) including the active may be added to the composition, or the composition may be “added” to the device (such as a tablet, capsule, or overlay) by placing the composition in a container including the device. Thus, the term “added,” when used to refer to adding the device and composition, includes adding the device to the composition and adding the composition to the device.

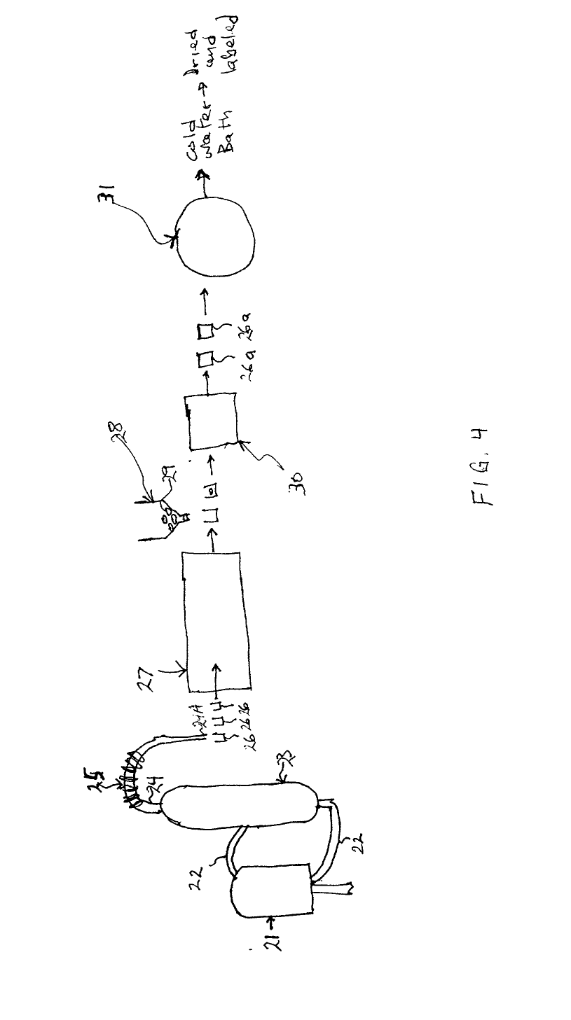

In FIG. 4, a potential method for practicing the invention is generally shown in block diagram form, but any method in which a composition is heated to or above the denaturing temperature of a relevant active may be used. The method depicted is generally the process for manufacturing enteral food, which is understood by persons who make this type of food. The composition is first added to a blender 21, which includes a mixing blade (not shown). Pipes or tubes 22 are for exchanging liquid composition between blender 21 and a mixing tank 23. The composition is generally formed in blender 21 by adding powdered ingredients to a liquid, such as water. The composition leaves tank 23 through a pipe or tube 24. Pipe 24 interacts with a heat exchanger 25 that heats the composition in pipe 24 to kill microbes that may be present. Composition leaving the end 24A of tube 24 is placed in open containers 26. Containers 26 are moved into an exhaustion tunnel 27 to remove air from the space in containers 26 above the level of the composition. The containers 26 then move past a pellet or tablet dispenser 28 containing tablets 29. One or more tablets 29 is placed into each container 26 and containers 26 are then moved into a lidder 30 that places lids on containers 26 to form sealed containers 26A. Sealed containers 26A pass into a heater to heat containers 26A in order to kill microbes. The containers are then preferably water-cooled, dried and labeled and sent to storage.

Tablets 29 are made in accordance with the invention and may be coated or uncoated. Further, capsules may be used in place of tablets, an overlay may be placed in container 26, or any other device may be utilized to add active(s) to the composition.

### EXAMPLE 1

In this example, all proportions are by weight, unless otherwise noted.

Valley Research, inc., located at P.O. Box 750, South Bend, Ind. 46624-0750, 1145 Northside Blvd., South Bend, Ind. 46615 (shipping address) makes powdered enzymes, including:

(1) A fungal enzyme called Valedase Fungal Lactase Concentrate, which is a food grade lactase enzyme (E.C. 3.2.1.23 beta-D-galactoside galactohydrolase) derived from the controlled fermentation of A. oryzae. The lactase catalyzes the hydrolysis of the lactose beta-D-galactoside linkage in lactose liberating one mole of D-glucose and one mole of D-galactose.

(2) A yeast-derived enzyme called Validase Yeast Lactase derived from the controlled fermentation of the yeast Kluyveromyces lactis. The lactase catalyzes the hydrolysis of the beta-D-galactoside linkage in lactase liberating one mole of D-glucose and one mole of D-galatose.

Either or both of these enzymes may be used to practice the invention. One problem with fungal enzyme is that it sometimes has some protease enzyme associated with it. Protease enzymes can break down proteins causing a bad flavor in the composition. But, the fungal enzyme would not create a bad flavor if purified to remove the protease enzyme, and in some compositions flavor is not an important feature. The fungal enzyme is acid resistant and it tends to last longer in acidic compositions and manufacturing processes. Therefore, for most compositions the fungal enzyme would be preferred because it would be more stable over the long term under acidic conditions. For a less acidic composition the yeast enzyme could be used.

A hand operated pill compactor, which is known to people skilled in the art, and is basically a press having a lever for making compressed tablets, was used to make experimental tablets. The compactor utilized can make tablets of about 10-15 millimeters in diameter and of varying lengths, depending on how much material is added. A fungal enzyme powder mixture was placed into the pill compactor, and then the lever arm was pressed to compress the powder and create a cylinder-shaped tablet approximately ¼″ in diameter and ¼″ in length. The tablet was comprised of approximately 1% fungal enzyme thoroughly mixed with approximately 94% by weight of malodextrin and 5% steeric acid (used to adhere the powered ingredients). Only a very small amount of enzyme is required and because of the cost it is preferred that only a small amount be used. But, pure enzyme could have also been used. Two tablets of approximately equal size were made using this method.

A coating was made by mixing sugar (sucrose) mixed with just enough water to dissolve it, boiling the mixture (using a candy thermometer, the mixture was heated to “hard rock candy” temperature) to create a syrup and then removing it from the heat. The syrup was then poured onto a cookie tray and rolled to form a sheet while still warm. A sliver of about 1/16″ thick was then cut from the sheet and one of the tablets was rolled in the sliver by hand to create a coating of about 1/16″-⅛″ around the tablet. The coated tablet was then allowed to sit overnight.

The two tablets were then tested. Each of the tablets was placed into a respective beaker. Approximately 300 ml of composition of a 10% lactose solution by weight in water was heated to 190° F., which is the temperature to which enteral food compositions are normally heated prior to being packaged, and a temperature that would normally denature the enzyme. The composition was then added to each of the two beakers. The composition in the beaker including the uncoated tablet is referred to as composition #1, and the composition in the beaker including the coated tablet is referred to as composition #2.

The uncoated tablet just disintegrated in composition #1; it did not dissolve slowly. The coated tablet did not immediately disperse into solution in composition #2. Instead it appeared to stay intact and dissolve over time. Each of the compositions was then allowed to stand overnight at room temperature.

When a glucose test strip was placed in composition #1 the following day, no color change was noted. The composition did not taste sweet at all, indicating that the enzyme became denatured, since no apparent conversion of lactose to glucose had occurred. When a glucose test strip was placed in composition #2, it changed color indicating the presence of glucose (note: the sugar coating itself contained sucrose). Composition #2 tasted sweet, indicating that at least some of the enzyme had not been denatured and was converting the lactose to glucose.

Having now described preferred embodiments of the invention, modifications and variations might occur to others. The invention is thus not limited to the description of the preferred embodiments, but is instead set forth in the following claims and legal equivalents thereof. Additionally, unless stated otherwise, method steps may be performed in any order capable of rendering a composition including a beneficial effect.

What is claimed is:

1. A method for adding a heat-sensitive active material, such as an enzyme or antibody, to a composition, the method including the steps of:
(a) providing a tablet including the active, the tablet being coated;
(b) placing the composition into a container;
(c) adding the tablet to the container; and
(d) heating the container including the composition and the tablet to, or above, a temperature capable of denaturing the active;
whereby the tablet dissolves slowly enough that at least some of the active is not denatured and imparts a beneficial effect to the composition.

2. A method for adding a device containing an active to a food or cosmetic composition, the method including the steps of:
(a) heating a composition to or above a temperature capable of denaturing the active; and
(b) adding the device to the composition.
whereby the device dissolves slowly enough in the composition so that at least some of the active is not denatured and imparts a beneficial effect to the composition.

3. The method of claim 2 wherein the active is an enzyme.

4. The method of claim 2 wherein the composition is heated to or above the denaturing temperature after the device is added to the composition.

5. The method of claim 2 wherein the device is added to the composition when the composition is at or above the denaturing temperature.

6. The method of claim 2 that further comprises the steps of placing the composition into a container and sealing the container.

7. The method of claim 6 wherein the composition is added to the container while the composition is at or above the denaturing temperature.

8. The method of claim 6 wherein the device is added to the composition after the composition is placed in the container.

9. The method of claim 6 wherein the device is added to the container prior to the composition being placed in the container.

10. The method of claim 2 wherein the active is an antibody.

11. The method of claim 3 wherein the active is a lactose-converting enzyme.

12. The method of claim 2 wherein the active is a vitamin.

13. The method of claim 2 wherein the device includes an exterior coating.

14. The method of claim 2 wherein the composition is a food.

15. The method of claim 11 wherein the beneficial effect is the conversion of lactose into another substance.

16. The method of claim 15 wherein 70% or more of the lactose is converted.

17. The method of claim 15 wherein 90% or more of the lactose is converted.

18. The method of claim 15 wherein 95% or more of the lactose is converted.

19. The method of claim 15 wherein 99% or more of the lactose is converted.

20. The method of claim 13 wherein the coating is a sugar coating.

21. The method of claim 2 wherein the denaturing temperature is 180° F. or higher.

22. The method of claim 14 wherein the composition is an enteral food.

23. The method of claim 2 wherein the device is a tablet.

24. The method of claim 2 wherein the device is an overlay.

25. A method for adding a lactose-converting active to a lactose-containing composition, and thereby converting at least some of the lactose in the lactose-containing composition to another substance, the method comprising the steps of:
(a) heating the lactose-containing composition to or above a temperature that would destroy or denature the active; and
(b) adding a device including the lactose-converting active to the lactose-containing composition, the device temporarily preventing at least some of the lactose-converting active from coming into direct contact with the lactose-containing composition, in order to enable at least some lactose-converting active to not be denatured and convert some of the lactose to another substance.

26. The method of claim 25 wherein the lactose converting substance is an enzyme.

27. The method of claim 25 wherein the device is a tablet.

28. The method of claim 27 wherein the tablet comprises an external coating.

29. The method of claim 27 wherein the tablet has an outer surface treated with gamma rays.

30. The method of claim 25 wherein the lactose-containing composition is heated to 180° F. or above.

31. The method of claim 25 wherein the device is added to the composition while the composition is at or above the temperature that would denature or destroy the active.

32. The method of claim 25 further comprising the step of cooling the lactose-containing composition.

33. The method of claim 32 wherein the cooling step occurs after the structure or device is added.

34. The method of claim 32 wherein the cooling step occurs before the device is added.

35. The method of claim 25 wherein the lactose-containing composition is heated to or above the temperature that would destroy or denature the active after the device is added.

36. The method of claim 25 that includes the further step of placing the lactose-containing composition into a container.

37. The method of claim 36 wherein the device is added to the lactose-containing composition after the lactose-containing composition is placed in the container.

38. The method of claim 36 wherein the device is in the container prior to the lactose-containing composition being placed into the container.

39. The method of claim 36 further comprising the step of sealing the container after the device and the lactose-containing composition are added.

40. The method of claim 25 wherein the lactose-containing composition is an enteral food.

41. The method of claim 40 wherein the lactose-containing composition has a pH of 6.0 or less.

42. A device for imparting a beneficial effect to a composition, the device comprising an active, and a material covering or coating at least part of the active, the device for being used in a composition that is heated to or above a temperature that would denature the active, the device dissolving slowly enough in the composition to prevent at least some of the active from being denatured, thus enabling the active to impart a beneficial effect to the composition.

43. The device of claim 42 wherein the material at least partially covering or coating the active prevents at least some of the active from directly contacting the composition while the composition is at a temperature sufficient to denature the active.

44. A tablet for use in converting lactose to another substance, the tablet comprising a lactose-converting substance and an exterior coating.

45. The tablet of claim 44 wherein the exterior coating is a sugar coating.

46. The tablet of claim 44 that further includes an outer surface, the outer surface being treated with gamma radiation.
